(19) United States
(12) Patent Application Publication
(10) Pub. No.: US 2012/0225911 A1
(43) Pub. Date: Sep. 6, 2012

(54) Replenishing body stores of fat-soluble vitamins A, and D eradicate chronic viral infection

(75) Inventor: Norman Smith, New Orleans, LA (US)

(73) Assignee: Norman A. Smith

(21) Appl. No.: 12/930,770

(22) Filed: Mar. 1, 2011

(51) Int. Cl. A61K 31/455; A61P 31/12

(52) U.S. Cl. 514/356

(57) ABSTRACT

The invention relates to beta-carotene and its' related analogues, zinc, vitamin D3, and niacin to enhance the performance of the innate and adaptive immune system. Beta-carotene and its' related analogues are converted to retinoic acid that activates retinoic acid receptors, retinoid X receptors and niacin activates the retinoid X receptors and peroxisome-proliferator-activated receptors to increase transcription activities or high density lipids(HDL). Zinc is required for transport of retinol and its conversion to retinal. Vitamin D activates transcription in activated immune cells. The overall effect of the composition is to improve the response of the immune system to effectively remove viral, bacterial, and other invading pathogens.

### FIELD OF THE INVENTION

The present invention relates to method for curing a condition in a subject by enhancement or sustaining the retinoid signaling pathway in said subject. The invention further relates to human evolution conserved immune system to eradicate foreign pathogens from the body.

### BACKGROUND TO THE INVENTION

Beta-carotene (pro-vitamin A) and vitamin A (retinol or all-trans retinol) are metabolized to retinoid derivatives which function to activate gene transcription for lymphocyte differentiation and proliferation (Mora, Iwata and von Andrian, 2008). All-trans-retinoic acid and 9-cis-retinoic acid activate retinoic acid receptors (RARs) and whereas retinoid X receptors (RXRs) is only activated by 9-cis-retinoic acid (Kastner et al 1994; Kliewer et al, 1994).

Foreign pathogens have been a consent challenge to human health. These invaders establish infections either intra or extracellular to hijack nutrients and support its reproduction. Some have developed strategies, and chemical factors to evade or any alter the immune response.

The current art of pharmaceuticals has not been able to eradicate such infections as HSV, S. aureus, or malaria. However pharmaceuticals have made life better for those subjects with chronic infection. The methodologies used has been to exploit a step in the life cycle of the pathogen or of the immune pathway.

The present invention finds a method to enhance the immune system response beyond the current art achievements.

### SUMMARY OF THE INVENTION

The present invention corrects a deficiency of substrate, retinoic acid, for robust signaling in the retinoid pathway.

Beta-carotene is chiefly obtained from vegetables or lesser amount from animal materials (organs). Unfortunate, the usage of petroleum-based fertilizer disrupts the microbial ecology of the soil. The effect is a lower amount of available vitamins and minerals from the vegetables. Since the functionality of the immune system depends on the metabolism of vitamin A and D, the gradual loss has led to increase infection within industrialized nations.

The present invention provides a method to correct fat-soluble vitamins deficiency by oral consumption of naturally occurring beta-carotene and vitamin D3, and zinc and niacin also to enhance the immune response. These are also the components in the composition of the invention.

The following description is not to be construed as limiting, it being understood that the skilled person may carry out many obvious variations to the invention.

One object of the present invention is restoration of conserved evolutionary pathways to eradicate pathogens infection.

Another object of the present invention is the addition of zinc to limit or if not to prevent the “yellowish skin” due to beta-carotene, all-trans-retinal or 9-cis retinal, or vitamin A (retinol) consumption because of a zinc deficiency and/or low serum retinoic binding protein.

Another object of the present invention is avoidance of complications and side-effects associated with pharmaceuticals, current art.

### CASE STUDY

A male with chronic herpes simplex virus for 15 years was started on beta-carotene 75,000 I.U. dosed 3 times a day, vitamin D3 15,000 I.U. dosed 3 times a day, niacin 500 mg dosed 2 times a day, and zinc 50 mg dosed 3 times a day. The subject followed the regiment for 12 months. At the end of therapy, the subject was tested for blood levels of herpes simplex virus type 1 and 2. The laboratory result reported negative detection of the virus (report #1), and detected antibody to the virus (report #2).

The case study revealed that chronic infection persists in a weaken immune system. At dosages beyond the recommended daily requirement, the body maintains homeostasis of converting inactive vitamins to its active form for storage and metabolism.

The study also validates the capacity of human evolutionary conserved immune system to effectively eradication viral infection and its potential for eradication of other foreign invaders.

1. A method for treating/eradicating invading pathogens by enhancing retinoic acid signaling pathways, peroxisome-proliferator-activated receptor, and vitamin D receptors activities wherein said conditions is a viral, bacterial, fungal, or helminthes infection.

2. (canceled)

3. A method according to claim 1 wherein said component of the retinoid signaling pathway is aldehyde dehydrogenase and/or short chain dehydrogenase/reductases.

4. (canceled)

5. A method according to claim 3 wherein said component retinoic acid activates retinoic acid receptors, and retinoid X receptors.

6. (canceled)

7. A method according to claim 1 wherein said components, vitamin D3, activate complex vitamin D receptor-retinoid X receptor.

8. A method according to claim 1 wherein said component zinc enhances retinoic binding protein synthesis.

9. (canceled)

10. A method according to claim 1 wherein said components directly affected the performance of the immune system.

11. (canceled)

12. A method according to claim 10 wherein said components metabolism level determine the outcome of the immune response.

13. (canceled)

14. A method according to claim 12 wherein eradication of said pathogen is achieved through adequate available components for metabolism for clearance of infection.

15. (canceled)

16. (canceled)
